(19) United States
(12) Patent Application Publication
(10) Pub. No.: US 2023/0048115 A1
(43) Pub. Date: Feb. 16, 2023

(54) METHOD AND APPARATUS FOR THE AUTOMATED TRANSFER OF AN INTRAOCULAR LENS

(71) Applicant: Alcon Inc., Fribourg (CH)

(72) Inventors: David Heemyung Kim, Frankfurt (DE); Christoph Ludwig, Brensbach (DE); Gerhard Klokow, Rheinfelden (DE)

(21) Appl. No.: 17/818,746

(22) Filed: Aug. 10, 2022

Related U.S. Application Data
(60) Provisional application No. 63/233,420, filed on Aug. 16, 2021.

(51) Int. Cl. B65G 49/05; A61F 2/16; B25J 15/06; B25J 15/00; B25J 9/16; B25J 13/08; B65G 47/91; F16D 27/01; F16D 27/14

(52) U.S. Cl. CPC B65G 49/05; A61F 2/16; B25J 15/0658; B25J 15/0028; B25J 9/1697; B25J 13/08; B65G 47/91; F16D 27/01; F16D 27/14; A61F 2002/1683; A61F 2240/00

(57) ABSTRACT

Disclosed is a method for the automated transfer of an intraocular lens (1) comprising an optical lens body (10) and two haptics (11) attached to a peripheral edge of the optical lens body (10) and extending outwardly from the peripheral edge of the optical lens body (10). The method comprises the steps of:
picking the intraocular lens (1) up at a start location;
moving the intraocular lens (1) to a destination location;
releasing the intraocular lens (1) at the destination location,
   wherein picking the intraocular lens (1) up at the start location comprises
gripping the intraocular lens (1) only at the haptics (11) of the intraocular lens (1).

### PRIORITY CLAIM

This application claims the benefit of priority of U.S. Provisional Patent Application Ser. No. 63/233,420 titled “METHOD AND APPARATUS FOR THE AUTOMATED TRANSFER OF AN INTRAOCULAR LENS,” filed on Aug. 16, 2021, whose inventors are David Heemyung, Christoph Ludwig and Gerhard Klokow, which is hereby incorporated by reference in its entirety as though fully and completely set forth herein.

### FIELD OF THE INVENTION

The present invention relates to the field of handling intraocular lenses (IOLs), and in particular to a method and apparatus for the automated transfer of an intraocular lens from a start location to a destination location.

### BACKGROUND

An intraocular lens—as its name says—is implanted into the eye of a patient, and in the vast majority of the cases an intraocular lens is used to replace the natural lens of the eye (e.g. in the treatment of cataract). An intraocular lens typically comprises an optical lens body (providing the required refractive power) and two haptics attached to the peripheral edge of the optical lens body and extending outwardly from the peripheral edge of the lens body. The purpose of the haptics is to properly position the intraocular lens including the optical lens body in the capsular bag of the eye of the patient. Obviously, therefore, the intraocular lens is a delicate medical article that must be treated with the utmost care possible during manufacture and handling, since—once implanted into the eye of the patient—the optical lens body of the intraocular lens providing the required refractive power must be free from any defects that may have a negative impact on the vision of the patient.

During manufacture and subsequent packaging, the intraocular lens must be transferred between different stations/locations. For example, the intraocular lens must be transferred from one carrier or fixture that carries the intraocular lens during a first manufacturing step to another carrier or fixture that carries the intraocular lens in a subsequent manufacturing step (e.g. from a carrier or fixture used during cleaning the intraocular lens to a carrier or fixture used during inspection of the intraocular lens). Many of the handling/transfer processes are presently carried out more or less manually by operators using specific tools such as for example a specific tool (similar to a forceps) allowing to carefully grip the intraocular lens at the peripheral edge of the optical lens body in order to avoid scratches or other damages to the optical lens body, as scratches or other damages in the optical lens body may lead to rejection of the intraocular lens during inspection. However, these manual handling/transfer processes are cumbersome for the operator, and even if the operator applies the utmost care the various intraocular lenses are not always placed on the carrier or fixture in the same position, as this is simply impossible in a manual procedure. Accordingly, from the point of view of repeatability and reliability as well as from the point of view of efficiency manual processes are disadvantageous.

Another problem occurring with an intraocular lens made of a flexible material is that the haptics may get slightly flexed or bent during the manufacturing process. At the time of inspection, however, not only the optical lens body is inspected for its integrity and compliance with the specifications, but also the overall dimensions of the intraocular lens including the haptics are measured and it is determined whether the overall dimensions of the intraocular lens meet the predefined specifications. In case the haptics are flexed or bent during manufacturing so that they do not have their regular orientation, this may lead to rejection of an intraocular lens during inspection (due to not meeting the specifications as regards the overall dimensions of the intraocular lens) although the intraocular lens would be compliant with the specifications if the haptics were properly oriented.

It is therefore an object of the invention to suggest a method and an apparatus overcoming the afore-mentioned disadvantages.

### SUMMARY OF THE INVENTION

The present invention overcomes the afore-mentioned disadvantages by suggesting a method and an apparatus as it is specified by the features of the respective independent claim. Advantageous aspects of the method and apparatus are specified in the respective dependent claims.

In particular, the method for the automated transfer of an intraocular lens comprising an optical lens body and two haptics attached to a peripheral edge of the optical lens body and extending outwardly from the peripheral edge of the optical lens body comprises the steps of:
picking the intraocular lens up at a start location;
moving the intraocular lens to a destination location;
releasing the intraocular lens at the destination location,
   wherein picking the intraocular lens up at the start location comprises
gripping the intraocular lens only at the haptics of the intraocular lens.

According to one aspect of the method according to the invention, gripping the intraocular lens only at the haptics is performed using a gripper comprising aspiration ports to attach the intraocular lens to the gripper only at the haptics of the intraocular lens by positioning the aspiration ports of the gripper adjacent to the haptics of the intraocular lens and applying vacuum to the aspiration ports. Releasing the intraocular lens from the gripper at the destination location is performed by applying overpressure to the aspiration ports to detach the haptics of the intraocular lens from the aspiration ports.

According to a further aspect of the method according to the invention, the aspirations ports of the gripper comprise two aspiration ports arranged at a distal end of the gripper and projecting distally away from the gripper. Each of the two aspiration ports comprises at its distal end an aspiration opening surrounded by a lens attachment surface. According to this aspect, the method further comprises
positioning the distal end of one aspiration port of the two aspiration ports adjacent to one of the two haptics of the intraocular lens such that the aspiration opening of that one aspiration port is covered by a portion of the one of the two haptics arranged adjacent thereto,
positioning the other aspiration port of the two aspiration ports adjacent to the other of the two haptics of the intraocular lens such that the aspiration opening of that other aspiration port is covered by a portion of the other one of the two haptics, and
applying the vacuum to the aspiration openings of the two aspiration ports to attach the one of the two haptics to the lens attachment surface of the one aspiration port and the other of the two haptics to the lens attachment surface of the other aspiration port.

In one aspect of the method according to the invention the gripper is a gripper having the two aspiration ports fixedly arranged at the distal end of the gripper.

In another aspect of the method according to the invention, the gripper is a gripper having the two aspiration ports rotatably arranged at the distal end of the gripper. Each of the two aspiration ports is rotatably arranged about a respective predetermined rotational axis which is normal to a plane defined by the lens attachment surfaces of the two aspiration ports.

According to yet a further aspect of the method according to the invention the method further comprises
prior to applying the vacuum to the aspiration openings of the two aspiration ports, determining an actual rotational orientation of each of the two haptics of the intraocular lens,
rotating each of the two aspiration ports about the respective predetermined rotational axis until the rotational orientation of the one aspiration port corresponds to the determined actual rotational orientation of the one of the two haptics and the rotational orientation of the other aspiration port corresponds to the determined actual rotational orientation of the other of the two haptics, and thereafter
applying the vacuum to the aspiration openings of the two aspiration ports to attach the one of the two haptics to the lens attachment surface of the one aspiration port and the other of the two haptics to the lens attachment surface of the other aspiration port.

Still in accordance with a further aspect of the method according to the invention the method further comprises
in case the actual rotational orientation of the one of the two haptics or of the other of the two haptics of the intraocular lens deviates from a respective predetermined rotational orientation:
   rotating the one aspiration port with the one of the two haptics attached to the lens attachment surface thereof and/or the other aspiration port with the other of the two haptics attached to the lens attachment surface thereof about the respective predetermined rotational axis until each of the two haptics has the predetermined rotational orientation, and
   at the destination location, applying the overpressure to the aspiration openings of the two aspiration ports with each of the two haptics having the predetermined rotational orientation.

The apparatus for the automated transfer of an intraocular lens comprising an optical lens body having a peripheral edge as well as two haptics attached to the peripheral edge of the optical lens body and extending outwardly from the peripheral edge of the optical lens body, comprises a gripper, and the gripper includes:
pressure supply connectors for the supply of vacuum or overpressure;
two aspiration ports arranged at a distal end of the gripper and projecting distally away from the gripper, each of the two aspiration ports at a distal end thereof comprising an aspiration opening that is surrounded by a lens attachment surface,
two separate fluid channels, each of the two separate fluid channels fluidically connecting a respective one of the two aspiration ports with the pressure supply connectors for the supply of vacuum or overpressure so as to form two separate fluidic connections between the pressure supply connectors and the aspiration openings of the aspiration ports.

The two aspiration ports are spaced from one another by a distance ranging from 5 mm to 17 mm (millimeters) measured in a plane defined by the lens attachment surfaces, for gripping the intraocular lens only at the haptics by attaching the haptics of the intraocular lens to the lens attachment surfaces of the aspiration ports, and for releasing the intraocular lens by detaching the haptics of the intraocular lens from the lens attachment surfaces.

According to one aspect of the apparatus according to the invention, the two aspiration ports are fixedly arranged at the distal end of the gripper.

According to another aspect of the apparatus according to the invention, the two aspiration ports are rotatably arranged at the distal end of the gripper. Each of the two aspiration ports is rotatably arranged about a respective predetermined rotational axis which is normal to the plane defined by the lens attachment surfaces.

In accordance with a further aspect of the apparatus according to the invention, the gripper further comprises two independent rotary motors, a first rotary motor and a second rotary motor each having a rotary drive shaft. The rotary drive shaft of the first rotary motor is connected by a torque-proof connector with a first gripper finger having one of the two aspiration ports arranged at a distal end thereof, and the rotary drive shaft of the second rotary motor is connected by a further torque-proof connector with a second gripper finger having the other one of the two aspiration ports arranged at a distal end thereof.

In accordance with still a further aspect of the apparatus according to the invention, each of the torque-proof connector and the further torque-proof connector comprises a magnetic clutch including a permanent magnet and two pins made of a magnetically susceptive material. The permanent magnet is mounted in a torque-proof manner to a distal end of the rotary drive shaft and faces towards a proximal end of the respective one of the first or second gripper fingers. The two pins are arranged at the proximal end of the respective one of the first or second gripper fingers and face towards the permanent magnet. The distal ends of the two pins are fixedly connected with the respective one of the first or second gripper fingers, and the proximal ends of the two pins are magnetically coupled to the permanent magnet.

In accordance with yet a further aspect of the apparatus according to the invention, the first gripper finger comprises an abutment flange arranged immediately proximal to the one aspiration port and the second gripper finger comprises a further abutment flange arranged immediately proximal to the other aspiration port. The gripper at its distal end comprises first and second abutment projections projecting distally from the distal end of the gripper at opposite sides of the gripper. The first abutment projection forms a stop for the abutment flange of the first gripper finger to define a predetermined rotational orientation of the one aspiration port when the abutment flange abuts against the first abutment projection, and the second abutment projection forms a stop for the further abutment flange of the second gripper finger to define a predetermined rotational orientation of the other aspiration port when the further abutment flange abuts against the second abutment projection.

According to a further aspect of the apparatus according to the invention, the apparatus further comprises an illumination source for illuminating an intraocular lens carried by a lens carrier as well as a camera for capturing an image of the illuminated intraocular lens carried by the lens carrier to determine the position of the intraocular lens and the rotational orientation of the haptics of the intraocular lens carried by the lens carrier. The apparatus further comprises a control unit coupled to the camera and to the gripper, for moving the gripper with the two aspiration ports to a position in which the two aspiration ports are arranged adjacent to the haptics of the intraocular lens such that the rotational orientation of the two aspiration ports corresponds to the determined actual rotational orientation of the two haptics of the intraocular lens.

Yet in accordance with a further aspect of the apparatus according to the invention, the apparatus further comprises a support plate comprising a plurality of mounting locations for mounting different types of lens carriers thereto. The apparatus further comprises at least two different lens carriers of different types mounted to the mounting locations. The support plate, the mounting locations and the at least two lens carriers of the different types are configured such that an intraocular lens arranged on a said lens carrier, regardless of the type of lens carrier, is arranged in the same plane parallel to the plane defined by the lens attachment surfaces of the aspiration ports.

The method according to the invention is advantageous in that it is an automated method which can be performed with high repeatability and reliability. In particular, the IOL can be picked up by gripping it at the two haptics only and repeatably placing it at the destination location (e.g. on a carrier) in the same manner and at the same position. Thus, the IOL is not gripped at the optical lens body at all (not even at the peripheral edge thereof) so that scratches or any damages to the optical lens body of the IOL may be completely avoided.

In case gripping the IOL is performed with the aid of a gripper comprising aspiration ports, the aspiration ports may be positioned adjacent to the haptics of the IOL and vacuum may be applied to the aspiration ports to attach the IOL to the aspiration ports (more precisely: to the lens attachments surfaces of the aspiration ports) only at the haptics. Releasing the IOL from the gripper at the destination location may be performed through the application of overpressure to the aspiration ports in order to detach the haptics of the IOL from the aspiration ports (more precisely: from the lens attachment surfaces of the aspiration ports). This enables a very careful handling of the IOL without any contact of the gripper to the optical lens body.

For example, a gripper with two aspiration ports may be used, each of which comprises at its distal end an aspiration opening which is surrounded by a lens attachment surface. The distal end of one aspiration port is then positioned adjacent to one of the two haptics, and the distal end of the other aspiration port is positioned adjacent to the other of the two haptics. The aspiration ports are positioned such that the aspiration opening of each aspiration port is covered by a portion of the respective haptic. The term “covered” in this regard does not mean that the IOL is already attached to the lens attachment surface surrounding the aspiration opening, nor does it mean that the aspiration opening is fully covered by the portion of the haptics. Rather, the term “covered” is intended to describe a certain overlap of the portion of the respective haptic with the respective aspiration opening which is at least 50%.

Vacuum is then applied to the aspiration openings to make the haptics attach to the lens attachment surfaces surrounding the lens attachment openings. And although this may lead to scenarios in which the aspiration opening of an aspiration port is not fully covered by the portion of the respective haptic after the gripping action, despite a small leakage stream the respective haptic remains securely attached to the respective lens attachment surface. Ideally, however, the aspiration openings are fully covered by the portion of the respective haptic, so that once the IOL is attached to the gripper, there is no leakage stream at all resulting in a strong attachment of the haptics to the lens attachment surfaces.

Generally, the haptics are attached to the optical lens body at well-defined locations and with a well-defined (desired) orientation. Accordingly, it is possible to use a gripper in which the aspiration ports are fixedly arranged at the distal end of the gripper (in a position such that the aspiration ports have a distance and orientation that corresponds to the said well-defined location and orientation of the haptics). It is thus possible to reliably pick the IOL up with a gripper that is comparatively simple from a constructional point of view.

Alternatively, a gripper may be used in which the two aspiration ports are rotatably arranged at the distal end of the gripper. Each aspiration port is rotatable about a respective predetermined rotational axis which is normal to a plane defined by the lens attachment surfaces of the aspiration ports. Such a gripper—while being more complex from a constructional point of view—may account for deviations of the actual location and orientation of the haptics from the said well-defined (desired) location and orientation.

When using such a gripper, it is possible to first determine the actual location and orientation of each of the two haptics of the IOL, then rotate each of the aspiration ports about the predetermined rotational axis such that there is the greatest possible overlap of the aspiration openings with the respective portions of the haptics. Only thereafter, vacuum is applied to the aspiration openings to make the haptics of the IOL attach to the lens attachment surfaces of the aspiration ports with the greatest overlap possible, ideally with the haptics fully covering the aspiration openings.

As regards the apparatus according to the invention, the apparatus is advantageous as it is able to grip the IOL by attaching the IOL to the gripper only at the haptics through the application of vacuum to the aspiration openings of the aspiration ports. To achieve this, vacuum is supplied to the pressure supply connectors from which the vacuum is further supplied to the aspiration openings of the two aspiration ports at the distal end of the gripper with the aid of two separate fluid channels. The haptics of the IOL (or to be more precise: portions of the haptics) are then attached to the lens attachment surfaces surrounding the aspiration openings of the aspiration ports. When the IOL is to be detached from the gripper, overpressure is supplied to the pressure supply connectors from which the overpressure is further supplied to the aspiration openings of the aspiration ports, thus detaching the IOL from the gripper (the IOL is carefully blown off). The aspiration ports are spaced from one another by a distance ranging from 5 mm to 17 mm, and is typically in the range of 9 mm to 10 mm.

The two aspiration ports may be fixedly arranged at the distal end of the gripper, i.e. with a fixed distance between the aspiration ports. As already mentioned, generally the haptics are attached to the optical lens body at well-defined locations and with a well-defined (desired) orientation. The fixedly arranged aspiration ports are then arranged at a fixed distance that corresponds to the distance between those portions of the haptics of the IOL that should be sucked against the lens attachment surfaces of the aspiration ports. It is thus possible to reliably pick the IOL up with a gripper that is comparatively simple from a constructional point of view.

Alternatively, the two aspiration ports may be rotatably arranged at a distal end of the gripper, with each of them being rotatably arranged about a respective predetermined rotational axis which is normal to the plane defined by the lens attachment surfaces. Such a gripper—while being more complex from a constructional point of view—may account for deviations of the actual location and orientation of the haptics from the said well-defined (desired) location and orientation. It is then possible to first determine the actual location and orientation of each of the two haptics of the IOL, then rotate each of the aspiration ports about the predetermined rotational axis to the actual location and orientation of the haptics such that there is the greatest possible overlap of the aspiration openings with the respective portions of the haptics to be sucked against the lens attachment surfaces. Only thereafter, vacuum is applied to the aspiration openings to make the haptics of the IOL attach to the lens attachment surfaces of the aspiration ports with the greatest overlap possible, ideally with the haptics fully covering the aspiration openings.

For the determination of the actual location and orientation of the two haptics of the IOL, the apparatus may comprise an illumination source (e.g. configured for dark-field illumination) for illuminating an IOL carried by a lens carrier. The apparatus may further comprise a camera for capturing an image of the illuminated IOL. From this image of the IOL the actual position and orientation of the two haptics of the IOL carried by the lens carrier can be determined (e.g. through image analysis). The apparatus further may comprise a control unit which is coupled to the camera and to the gripper. Depending on the position and orientation determined from the image of the IOL captured by the camera the control unit moves the gripper with the two aspiration ports to a position in which the two aspiration ports are arranged adjacent to the haptics of the IOL. In case the gripper comprises the afore-mentioned rotatably arranged aspiration ports, the aspiration ports may be rotated to the actual location and orientation of the haptics such that there is the greatest possible overlap of the aspiration openings with corresponding portions of the haptics, ideally with the haptics fully covering the aspiration openings. In case the gripper comprises the fixedly arranged aspiration ports, the gripper is moved to the position in which there is the greatest possible overlap of the aspiration ports with the corresponding portions of the haptics. As already mentioned, this may lead to scenarios in which the aspiration opening of an aspiration port is not fully covered by the portion of the respective haptic after the gripping action. However, even if there is a small leakage stream the respective haptic remains securely attached to the respective lens attachment surface.

To further increase repeatability and reliability, the apparatus may further comprise a support plate comprising a plurality of mounting locations to which different types of lens carriers may be mounted (e.g. lens carriers used for cleaning the IOLs and lens carriers used for the inspection of the IOLs). Two (or more than two) lens carriers of different types may be mounted to the mounting locations of the support plate, for example to transfer an IOL from one type of lens carrier (e.g. from a lens carrier for cleaning an IOL) to a different type of lens carrier (e.g. to a lens carrier for the inspection of an IOL). The support plate, the mounting locations and the different types of lens carriers are configured such that an IOL arranged on a said lens carrier, regardless of the type of lens carrier, is always arranged in the same plane parallel to the plane defined by the lens attachment surfaces of the aspiration ports. This allows for fixedly attaching the gripper to a robot that moves the gripper in a z-direction normal to the said plane (i.e. towards and away from the mounting plate) as well as in a plane parallel to the said plane. At least as regards the z-direction, the robot may then be taught not to move the gripper beyond a certain position in the z-direction to avoid collisions of the gripper with the IOL or with any of the lens carriers arranged on the support plate. Also, the robot may be taught what is the optimum position in the z-direction for picking up the IOL. These positions may be taught to the robot only once even if the gripper must be replaced, as long as the new gripper to be mounted has the same dimensions as the gripper to be replaced.

For rotating the two rotatably arranged aspiration ports, the gripper may comprise tow independent rotary motors, a first and a second rotary motor. Each of these first and second motors has a rotary drive shaft. The rotary drive shaft of the first motor is connected with a first gripper finger by a torque-proof connector. The first gripper finger has the first aspiration port arranged at a distal end of this first gripper finger. Likewise, the rotary drive shaft of the second motor is connected with a second gripper finger by a torque-proof connector. The second gripper finger has the second aspiration port arrange at a distal end of this second gripper finger. This allows to independently rotate the first and second gripper fingers, thereby rotating the respective aspiration ports, to a position in which the aspiration opening of the respective aspiration port has the greatest possible overlap with the corresponding portion of the respective haptic of the IOL. The torque-proof connection of the respective drive shaft of the first and second rotary motor allows for a very precise adjustment of the respective rotational position of the respective aspiration port arranged at the distal end of the first or second gripper finger.

Although many torque-proof connections are generally conceivable (including form-locking connections), one advantageous example for such torque-proof connection comprises a magnetic clutch that includes a permanent magnet and two pins made of a magnetically susceptive material (a magnetizable material). The permanent magnet may be mounted in a torque-proof manner to a distal end of the rotary drive shaft of the respective rotary motor and faces towards a proximal end of the respective one of the first or second gripper fingers. The two pins made of the magnetically susceptive material are arranged at the proximal end of the respective one of the first or second gripper fingers, too, and face towards the permanent magnet. The distal ends of the two pins are fixedly connected with the respective one of the first or second gripper fingers (for example, the distal ends of the two pins are press-fitted into corresponding holes), while the proximal ends of the two pins are magnetically coupled to the permanent magnet (for example, the proximal end faces of the two pins may abut against the distal end face of the permanent magnet). Such a magnetic clutch is simple from a constructional point of view while at the same time forming a reliable torque-proof connection. When the rotary drive shaft of the respective motor is rotated, the two pins are rotated, too, and due to being fixedly connected with the respective first or second griper finger, the two pins rotate the respective first or second gripper finger by exactly the same angle by which the rotary drive shaft is rotated. This allows for a very precise adjustment of the respective rotational position of the respective aspiration port arranged at the distal end of the first or second gripper finger.

The first gripper finger may additionally comprise an abutment flange which is arranged immediately proximal to the one aspiration port (of the two aspiration ports), and second gripper finger may comprise a further abutment flange which is arranged immediately proximal to the other aspiration port (of the two aspiration ports). The gripper, at its distal end, may comprise first and second abutment projections projecting distally from the distal end of the gripper at opposite sides. The first abutment projection forms a stop for the abutment flange of the first gripper finger to define a predetermined rotational orientation of the one aspiration port (of the two aspiration ports) when the abutment flange abuts against the first abutment projection. Likewise, the second abutment projection forms a stop for the further abutment flange of the second gripper finger to define a predetermined rotational orientation of the other aspiration port (of the two aspiration ports) when the further abutment flange abuts against the second abutment projection.

This represents a constructive measure for individually defining predetermined rotational orientations for the two aspiration ports. For example, each of the gripper fingers may be rotated to the positions in which its abutment flange abuts against the respective abutment projection. Starting from this predetermined orientation, the respective aspiration port can then be rotated to the desired rotational orientation in which the overlap of the aspiration opening of the respective aspiration port with the portions of the respective haptic of the IOL to be gripped is maximal. For example, these well-defined predetermined orientations may be stored in the control unit, so that upon having captured an image of the IOL and having determined the actual rotational orientation of the respective haptics of the IOL each of the first and second gripper fingers may be rotated to the actual rotational orientation of the respective haptic (starting from this well-defined predetermined orientation). This enables a very accurate adjustment of the desired orientation of the two aspiration ports.

Further advantageous aspects of the method and apparatus according to the invention will become apparent from the following description of embodiments of the invention with the aid of the schematic drawings.

### BRIEF DESCRIPTION OF THE DRAWINGS

FIG. 1 illustrates the process of a transfer of an intraocular lens (IOL) from a lens carrier used during manufacture of the IOL to a lens carrier used during inspection of the IOL;

FIG. 2 shows a perspective view of an embodiment of an apparatus for the automated transfer of an intraocular lens according to the invention;

FIG. 3 shows a scheme illustrating the transfer of an IOL from a respective start location to a respective destination location;

FIG. 4 shows a perspective view of some components of the embodiment of the apparatus of FIG. 2;

FIG. 5 shows an exploded perspective view of a first embodiment of a gripper of the apparatus according to the invention;

FIG. 6 shows a longitudinal section through the assembled gripper of FIG. 5;

FIG. 7 shows an enlarged view of the detail VII of FIG. 6;

FIG. 8 shows a perspective top view of an end piece of the gripper of FIG. 5;

FIG. 9 shows a perspective bottom view of the end piece of the gripper of FIG. 5;

FIG. 10 shows a bottom view of the end piece of the gripper of FIG. 5;

FIG. 11 shows a cross-sectional view of the end piece of the gripper of FIG. 5;

FIG. 12 shows a partially sectioned view of a portion of the end piece of FIG. 8;

FIG. 13 show the detail XIII of FIG. 11;

FIG. 14 shows an IOL gripped at its haptics by the first embodiment of the gripper;

FIG. 15 show a perspective top view of a portion of an inspection carrier during placement of the IOL by the first embodiment of the gripper;

FIG. 16 shows a perspective view of a second embodiment of a gripper of the apparatus according to the invention;

FIG. 17 shows a side view of the embodiment of the gripper shown in FIG. 16;

FIG. 18 shows a longitudinal section of the gripper along line XVIII-XVIII of FIG. 17;

FIG. 19 shows a longitudinal section of the gripper along line XIX-XIX of FIG. 18;

FIG. 20 shows a perspective view of the gripper finger of the gripper of FIG. 16;

FIG. 21 shows a side view of the gripper finger of FIG. 19;

FIG. 22 shows a longitudinal section of the gripper finger along line XXII-XXII of FIG. 21;

FIG. 23 shows the detail XXIII of FIG. 22;

FIG. 24 shows a top view of the gripper finger of FIG. 20;

FIG. 25 shows a bottom view of the gripper finger of FIG. 20;

FIG. 26 shows the detail XXVI of FIG. 25 in an enlarged view;

FIG. 27 shows a bottom view of the gripper of FIG. 16 with an IOL attached thereto that has a regular orientation of the haptics; and

FIG. 28 shows a bottom view of the gripper of FIG. 16 with an IOL attached thereto that has an irregular orientation of the haptics.

### DETAILED DESCRIPTION

As used in the specification including the appended claims, the singular forms “a”, “an”, and “the” include the plural, unless the context explicitly dictates otherwise. When using the term “about” with reference to a particular numerical value or a range of values, this is to be understood in the sense that the particular numerical value referred to in connection with the term “about” is included and is explicitly disclosed, unless the context clearly dictates otherwise. For example, if a range of “about” numerical value a to “about” numerical value b is disclosed, this is to be understood to include and explicitly disclose a range of numerical value a to numerical value b. Also, whenever features are combined with the term “or”, the term “or” is to be understood to also include “and” unless it is evident from the specification that the term “or” must be understood as being exclusive.

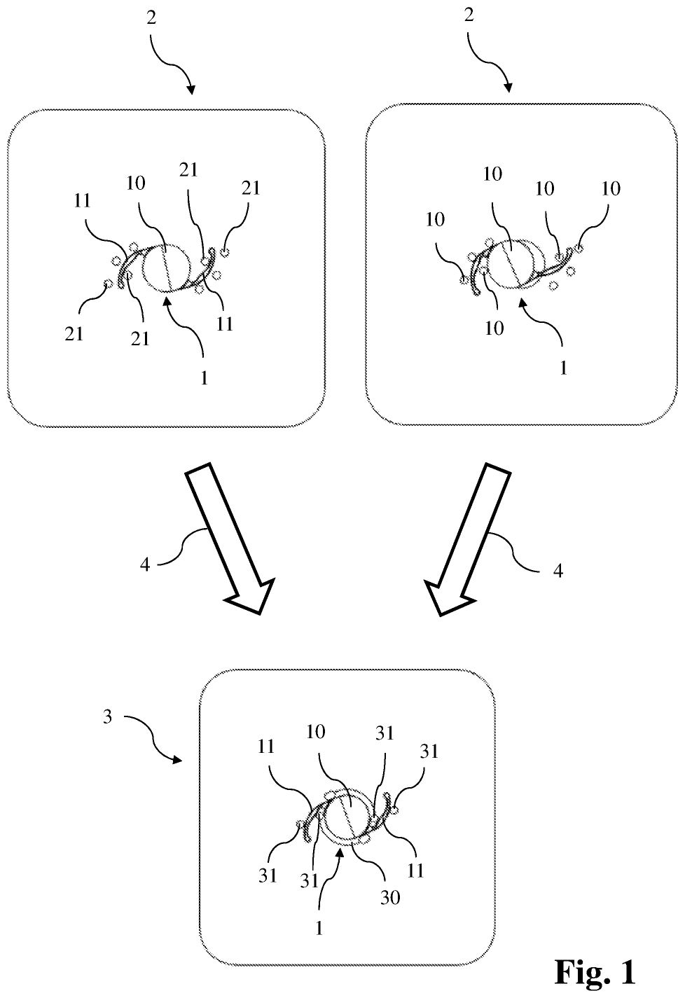

FIG. 1 illustrates the process of transferring an intraocular lens 1 (referred to as ‘IOL’ in the following) from a lens carrier 2 of a first type which may be used during manufacture of the IOL 1 to a lens carrier 3 of a second type (which may or may not be different from the first type) which may be used during inspection of the IOL 1. As can be seen in FIG. 1, IOL 1 comprises an optical lens body 10 as well as two haptics 11 extending outwardly from a peripheral edge of the optical lens body 10, as is conventional and well understood by those skilled in the art. Optical lens body 10 is a particularly delicate component of IOL 1 since optical lens body 10 is that component of IOL 1 generating the image on the retina of the eye after the IOL 1 has been implanted into the capsular bag of the eye of a patient, whereas the two haptics 11 serve for the proper arrangement of the IOL 1 as a whole, and of the optical lens body 10 in particular, in the capsular bag.

As mentioned further above already, not only the optical lens body 10 of the IOL 1 is inspected for its integrity and compliance with the specifications, but also the overall dimensions of the intraocular lens including the haptics are measured. By way of example, the diameter of the optical lens body 10 has a predetermined value which is a value ranging from 4.9 mm to 7.1 mm, and the overall diameter of the IOL 1 (distance between the tips of the two haptics 11) has a predetermined value which is typically in the range of 10.5 mm to 14.5 mm, but may even be larger than that. Only by way of example, the diameter of the optical lens body 10 may be 6.0 mm or 6.3 mm, and the overall diameter of the IOL 1 (including the haptics) may be 13.3 mm.

IOLs are typically made of materials exhibiting a high flexibility (e.g. silicone hydrogels), as during the surgical procedure it is desirable that the dimensions of the IOL 1 may be temporarily reduced to be as small as possible so that only a small incision is needed in the eye for the insertion of the IOL 1 into the capsular bag through the incision (scleral tunnel).

Due to the flexibility of the material the IOL 1 is made of, the haptics 11 may get slightly flexed or bent during the manufacturing process. To illustrate this, two such lens carriers 2 of the first type are shown in FIG. 1. The lens carrier 2 of the first type comprises some small pins 21 (or studs) for holding the IOL 1 in position on the lens carrier 2.

The haptics 11 of the IOL 1 on the lens carrier 2 shown on the left-hand side in FIG. 1 are arranged in their regular orientation relative to the optical lens body 10, whereas one of the two haptics 11 of the IOL 1 on the lens carrier 2 shown on the right-hand side in FIG. 1 is flexed or bent away from the optical lens body 10 while the other one of the two haptics 11 is flexed or bent towards the optical lens body 10. Typically, this flexing or bending of the haptic 11 means that the respective haptic 11 is slightly pivoted about the portion where the respective haptic 11 is attached to the lens body 10. And although the optical lens body 10 of IOL 1 shown on the lens carrier 2 on the right-hand side in FIG. 1 is displaced and the haptics 11 are flexed out of their regular orientation relative to the optical lens body 10, the dimensions of the IOL 1 are well within the specifications if the haptics 11 were arranged in their regular orientation relative to the optical lens body 10.

Accordingly, while a simple transfer of the IOL 1 from the lens carrier 2 of the first type shown on the left hand side in FIG. 1 to the lens carrier 3 of the second type used for the inspection of the IOL 1 may lead to a ‘pass’ (i.e. positive result) during the inspection (assuming the IOL 1 is free of any defects), the simple transfer of the IOL 1 from the lens carrier 2 shown on the right hand side in FIG. 1 to the lens carrier 3 may lead to a ‘reject’ (i.e. negative result) during the inspection due to the overall diameter of the IOL being outside the specifications. The latter result of the inspection is unwanted (as the IOL 1 has the proper dimensions if the haptics were properly oriented) and can be avoided.

Lens carrier 3 of the second type comprises some small pins 31 (or studs) for holding the IOL 1 in position on the lens carrier 3. Lens carrier 3 further comprises an inspection opening 30, and the IOL 1 is arranged on the lens carrier 3 such that the lens body 10 of IOL 1 is arranged above the inspection opening 30 to allow for the inspection of the lens body 10 of IOL 1 through this inspection opening 30. The process of transferring the IOL 1 from either the lens carrier 2 of the first type shown on the left-hand side in FIG. 1 or from the lens carrier 2 of the first type shown on the right-hand side in FIG. 1 to the lens carrier 3 of the second type is illustrated by a respective arrow 4.

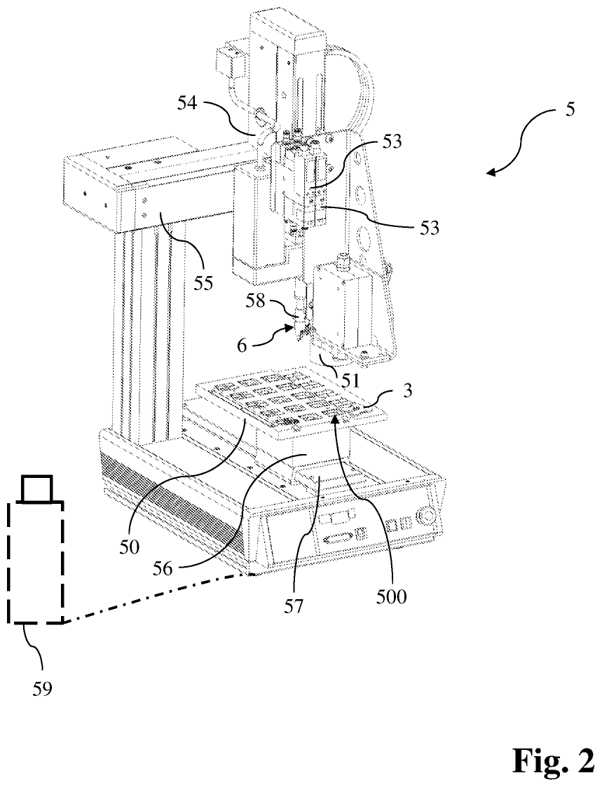

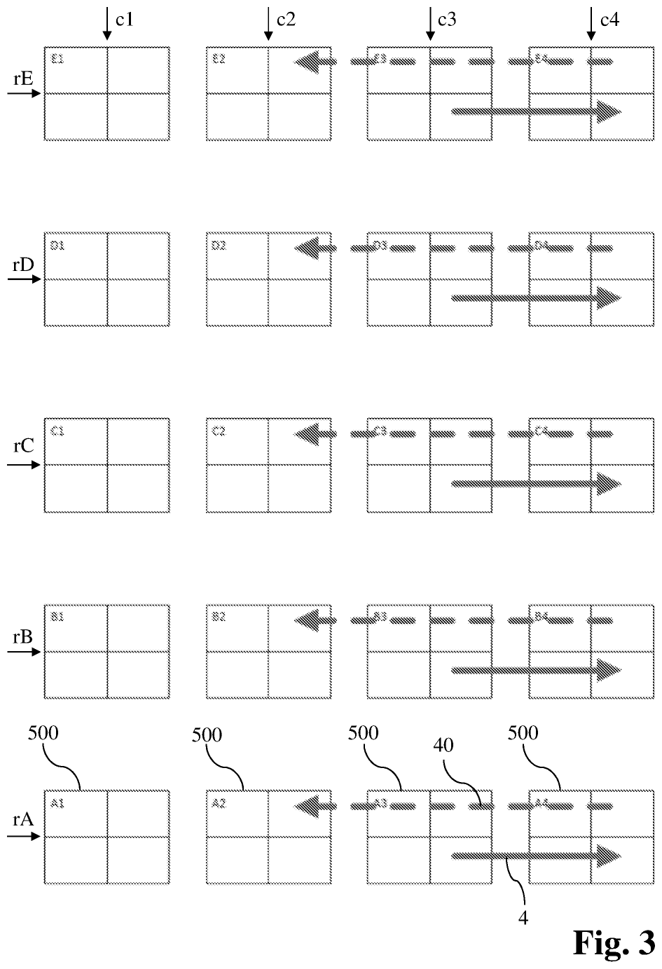

FIG. 2 shows a perspective view of an embodiment of an apparatus 5 for the automated transfer of the IOL 1. This embodiment of the apparatus 5 comprises a support plate 50 comprising a plurality of individual mounting locations 500 for mounting different types of lens carriers thereto. These mounting locations 500 are arranged in a matrix-like arrangement of rows and columns, as will be discussed in more detail with reference to FIG. 3. In FIG. 2 one lens carrier 3 of the second type is shown as being mounted to the support plate 5 at one of the mounting locations, while a lens carrier 2 of the first type (not shown in FIG. 2, see FIG. 1) may be mounted at that mounting location 500 to which the tip of the arrow points. In the embodiment shown, the individual mounting locations and the different types of lens carriers are configured such that, regardless of the type of lens carrier, an IOL 1 placed on such lens carrier is always arranged in the same plane (parallel to the plane of the support plate 50).

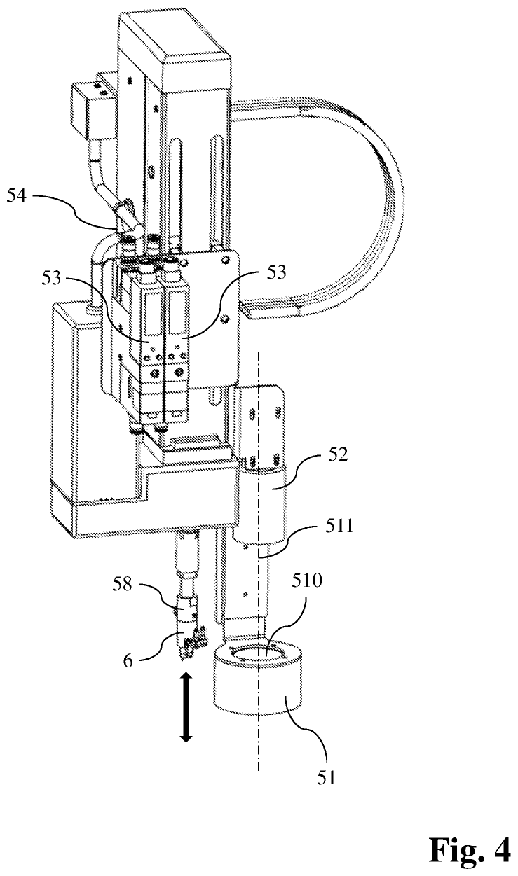

Apparatus 5 further comprises an illumination source 51, a camera 52 (not visible in FIG. 2, see FIG. 4), as well as control valves 53 for controlling the supply of either vacuum or overpressure. Apparatus 5 further comprises a gripper 6 for gripping the IOL 1 at the two haptics 11 of the IOL 1 only, and for transferring the IOL 1 from one type of lens carrier arranged on the support plate 50 to another type of lens carrier arranged on the support plate 50, as will be explained in more detail below. By way of example, the IOL 1 may be transported from a lens carrier 2 of the first type shown in FIG. 1 (used during manufacturing of the IOL 1) to a lens carrier 3 of the second type (used during inspection of the IOL 1).

The apparatus 5 may further comprise an additional camera 59 (schematically indicated in FIG. 2 by dashed lines) for confirming that the IOL 1 has been properly gripped by the gripper 6 and how the IOL 1 and the haptics 11 are actually attached to the gripper 6 prior to placing IOL 1 on the lens carrier 3 of the second type. This camera has a view from below, i.e. the view is from below towards the distal end of the gripper 6 to which the IOL 1 is attached. Thus, additional camera 59 may help in confirming that the haptics 11 are in the proper rotational orientation so that the IOL 1 can be reliably and successfully placed on the lens carrier 3.

A scheme illustrating the transfer of the IOL 1 from a respective start location to a respective destination location is shown in FIG. 3, and this illustrated scheme corresponds to the individual mounting locations 500 on the support plate 50 shown in FIG. 2. The individual mounting locations 500 are arranged along rows indicated by rA, rB, rC, rD and rE and columns indicated by c1, c2, c3, and c4. Accordingly, in the lowermost row of the scheme the individual mounting locations are denoted A1 (row A, column 1), A2, A3 and A4, while in the uppermost row of the scheme the individual mounting locations are denoted E1, E2, E3 and E4, the center of the respective mounting location 500 being indicated by crosshairs.

For example, in row rA the transfer of the IOL 1 (see FIG. 1) from the lens carrier 2 of the first type (see FIG. 1) which is arranged at mounting location A3 to the lens carrier 3 of the second type (see FIG. 1) which is arranged at mounting location A4 is indicated by the full arrow 4. After successful inspection, the IOL may be transferred from the lens carrier 3 of the second type at mounting location A4 to a lens carrier of a third type (storage carrier, not shown) arranged at mounting location A2, this being indicated by the dashed arrow 40.

Turning back to the apparatus 5 shown in FIG. 2, for performing the movement along the respective row, the gripper 6 and the other components (illumination source 51, camera 52, control valves 53) are mounted to a slide 54 which may be moved along a fixedly arranged arm 55 of apparatus 5. For a movement in the direction of the columns, the support plate 50 is mounted on a further slide 56 which is movable along a rail 57. Gripper 6 is separately movable (i.e. without the other components) towards and away from the support plate 50 in a direction normal to the plane defined by the support plate 50 on which the IOL 1 is arranged on the respective lens carrier 2 of the first type or on the lens carrier 3 of the second type (or on any further lens carrier).

FIG. 4 shows a perspective view of some components of the embodiment of the apparatus of FIG. 2 which are mounted to the slide 54, in particular the illumination source 51, the camera 52, the control valves 53 and the gripper 6. The illumination source 51 is an annular illumination source for emitting light downwardly towards the support plate 5 and has a central through-opening 510 extending in the longitudinal direction along an axis 511 and allowing the camera 52 to capture an image of the IOL 1 (not shown in FIG. 4) to be inspected through this through-opening 510. The IOL 1 to be inspected may thus be illuminated in a dark-field illumination configuration.

As mentioned, gripper 6 is movable in the direction towards and away from the support plate 50 (z-direction normal to the x-y-directions of the rows and columns), this direction being indicated by the double-headed arrow in FIG. 4. For that reason, gripper 6 is mounted to a mounting shaft 58 in a manner that will be described in more detail below. This mounting shaft 58 is arranged at a fixed predetermined lateral displacement (location in the x-y plane) relative to the axis 511. So once the location (in the x-y plane) of an IOL 1 to be picked up has been determined with the aid of the camera 52, due to the known lateral displacement of the mounting shaft 58 relative of the axis 511 the location to which the gripper 6 (which is mounted to the mounting shaft 58) must be moved to pick up the IOL 1 is exactly known to the apparatus 5. The gripper 6 mounted to mounting shaft 58 has a predetermined overall length, so that once mounted to the mounting shaft 58 it is only necessary to determine the lowermost position (location in the z-direction) to which he distal end of the gripper 6 may be moved once. At this lowermost position the distal end of gripper 6 (the aspiration ports for gripping the IOL 1, as will be explained below) is arranged shortly above the IOL 1 arranged on the respective lens carrier. The IOL 1 is always arranged in the same plane regardless of the type of lens carrier used, as has already been mentioned above. In this lowermost position the gripper 6 may pick up the IOL 1 from the respective lens carrier as will be described in more detail below. When the gripper 6 must be replaced, it is possible to simply unmount the gripper 6 from the mounting shaft 58 and to mount a new gripper 6 to the mounting shaft. As the new gripper 6 has the same dimensions as the old gripper 6, it is not necessary to teach the lowermost position of the gripper 6 to the apparatus again, but rather the already determined lowermost position can be maintained, so that the IOL 1 cannot get damaged at the time of being picked up. While the gripper is assigned reference numeral 6 in FIG. 2 and FIG. 4, in the following description of embodiments of the gripper reference numerals other than 6 are assigned to the gripper in order to be able to better distinguish the embodiments of the gripper.

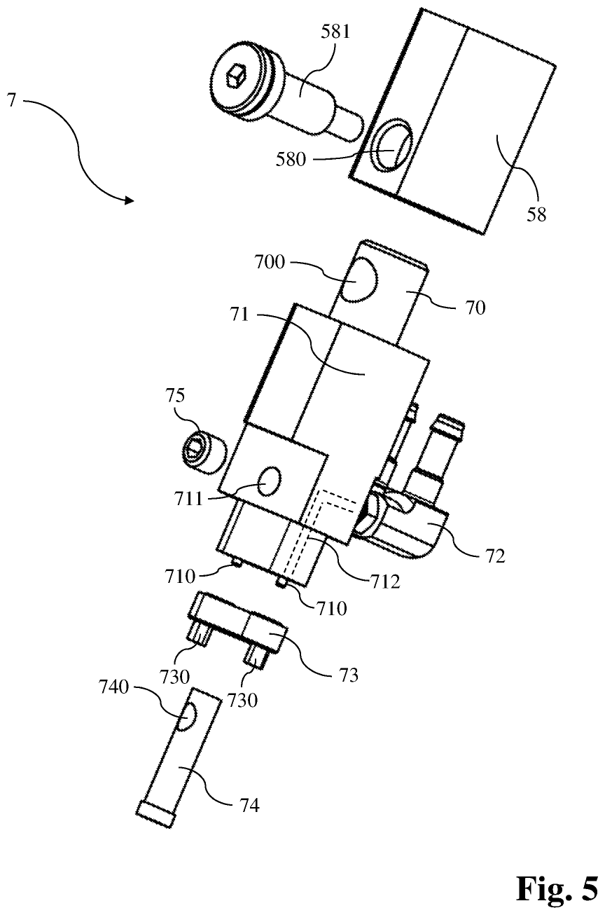

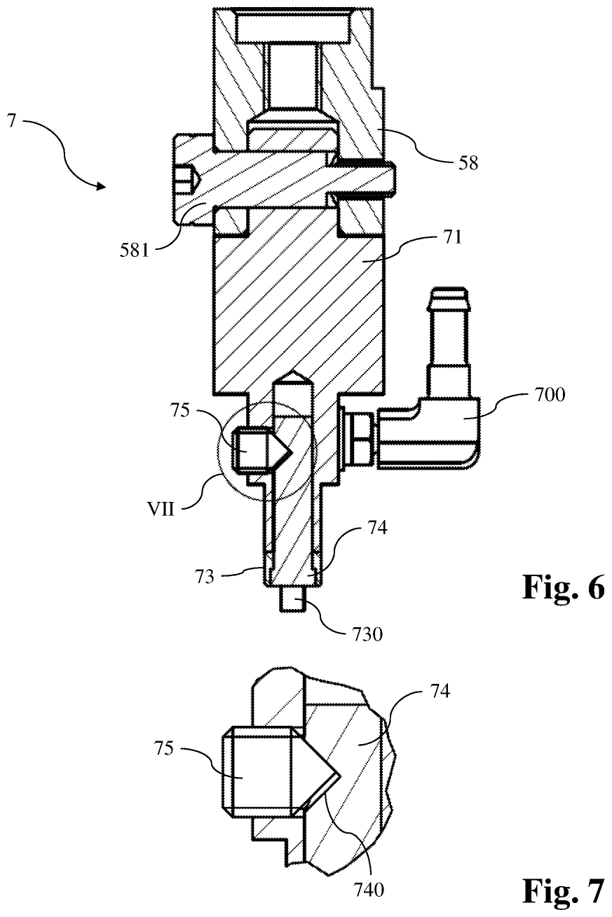

A first embodiment of the gripper 7 of the apparatus 5 according to the invention is described in the following with the aid of FIG. 5-FIG. 15. The first embodiment of gripper 7 shown in an exploded view in FIG. 5 comprises a mounting stud 70 having a through-hole 700 extending transversely through the mounting stud 70, so that the gripper 7 as a whole may be mounted to the mounting shaft 58 of the apparatus 5 with the aid of a fitting screw 581 extending through a through-hole 580 provided in the mounting shaft 58 of the apparatus 5 as well as through the through-hole 700 of the mounting stud 70 (see FIG. 6) of the gripper 7. Gripper 7 further comprises a gripper body 71 with two pressure supply connectors 72, e.g. small hose stems, which are mounted to the gripper body 71. Vacuum or overpressure may be alternatively supplied to each of the supply connectors 72. Gripper 7 further comprises a gripper end piece 73 with two aspiration ports 730, which is to be mounted to a distal end of the gripper body 71. To achieve a proper positioning of the end piece 73 relative to the gripper body 71 during mounting, two positioning pins 710 are arranged at the distal end of the gripper body 71 and are projecting distally away from the gripper body. The gripper end piece 73 is mounted to the gripper body 71 with the aid of a small tensioning bolt 74 having a conical notch 740 arranged at the proximal end of the tensioning bolt 74 for the engagement of a set screw 75 having a conical tip. The set screw 75 is screwed into a threaded through-bore 711 of gripper body 71 for engaging into the conical notch 740 of the tensioning bolt 74 to properly position the tensioning bolt 74, which in turn pulls the gripper end piece 73 against the gripper body 71. Two separate supply channels (i.e. supply channels which are not in fluid connection with one another) are provided in the interior of the gripper body 71, one such supply channel 712 being shown by dashed lines in FIG. 5. FIG. 6 shows the assembled gripper 7 mounted to the mounting shaft 58, and FIG. 7 shows in detail how the set screw 75 engages into the conical notch 740 of the tensioning bolt 74, pulling the tensioning bolt 74 upwardly to the desired position and thereby pulling gripper end piece 73 against gripper body 71.

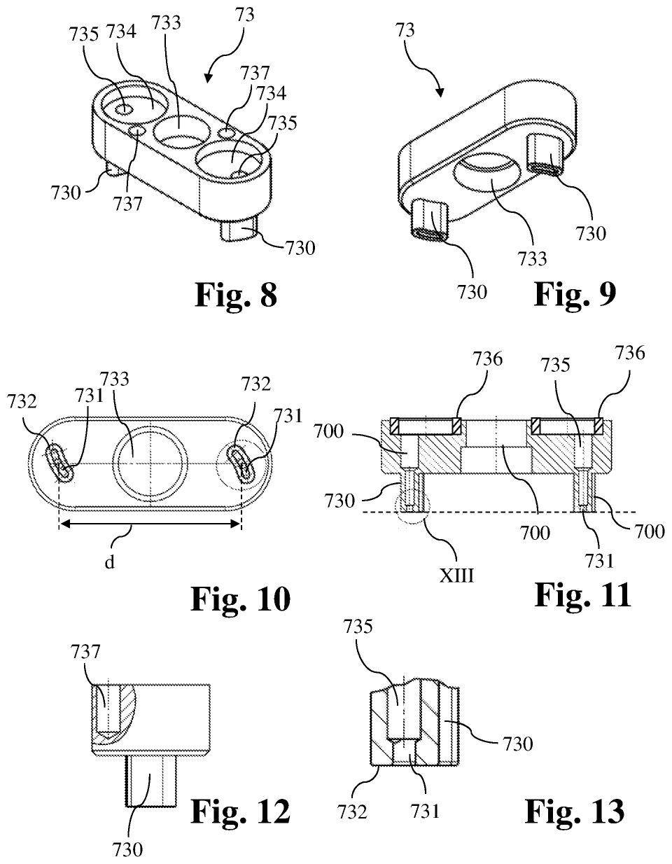

FIG. 8-FIG. 13 show the gripper end piece 73 in more detail. Gripper end piece 73 comprises the two (hollow) aspiration ports 730, each having an aspiration opening 731 which has the shape of a curved slot and is surrounded by a lens attachment surface 732. From the perspective top view shown in FIG. 8 it can be seen that gripper end piece 73 comprises two blind bores 737 (see also FIG. 12) for accommodating the two positioning pins 710 projecting distally away from the gripper body 71 in order to properly position the gripper end piece 73 relative to the gripper body 71. Gripper end piece 73 further comprises a central stepped through-bore 733 (see also FIG. 11) for accommodating the tensioning bolt 74 as well as two recesses 734. From the bottom of each of the recesses 734 a supply bore 735 extends down to the respective aspiration port 730 and opens into the aspiration opening 731 of the respective aspiration port 730. As can be seen in FIG. 10 already but can be seen even better in FIG. 11 and FIG. 13, the supply bore 735 is of circular cross-section and ends some axial distance away from the aspiration opening 731. Also, as indicated in FIG. 11 an O-ring 736 is arranged in each of the recesses 734, the respective O-ring 736 having an axial height such that it projects upwardly beyond the upper surface of the gripper end piece 73.

During mounting the gripper end piece 73 to the gripper body 71, the two positioning pins 710 projecting distally away from the gripper body 71 penetrate into the blind bores 737 of the gripper end piece 73, and once the set screw 75 starts engaging the conical notch 740 of tensioning bolt 74 the tensioning bolt 74 is pulled upwards thereby pulling the gripper end piece 73 against the gripper body 71. Through this pulling action, the two O-rings 736 are compressed so that two separate pressure-tight fluid channels are established, each pressure-tight fluid channel fluidically connecting one of the pressure supply connectors 72 with the aspiration opening 731 of a respective one of the aspiration ports 730 to either supply vacuum to the respective aspiration port 730 (for picking the IOL 1 up), or to supply overpressure to the respective aspiration port 730 (for releasing the IOL 1). Each of the pressure-tight fluid channels is formed by the supply channel 712 extending through the gripper body 71, and further by the recess 734 and the supply bore 735 extending from the bottom of recess 734 down to the aspiration opening 731 of the aspiration port 730.

As can be seen in FIG. 8-FIG. 13, the two aspiration ports 730 are fixedly arranged at the distal end of the gripper 7, or to be more precise at the distal end of gripper end piece 73 of gripper 7. The term ‘fixedly arranged’ means that a distance d between the two aspiration ports 730 (see FIG. 10) and the rotational orientation relative to one another cannot be changed. The distance d between the two aspiration ports has a value ranging from 5 mm to 17 mm, this distance d in any event being larger than the diameter of the optical body 10 of IOL 1 to be gripped, so that the IOL 1 may only be gripped at the haptics 11). More preferably, the value for this distance d ranges from 8 mm to 12 mm, and by way of example the value for the distance d may be 9.8 mm. The distance d is measured in a plane defined by the attachment surfaces 732 of the aspiration ports 730, this plane being indicated in dashed lines in FIG. 11 and extends normal to the plane of the drawing.

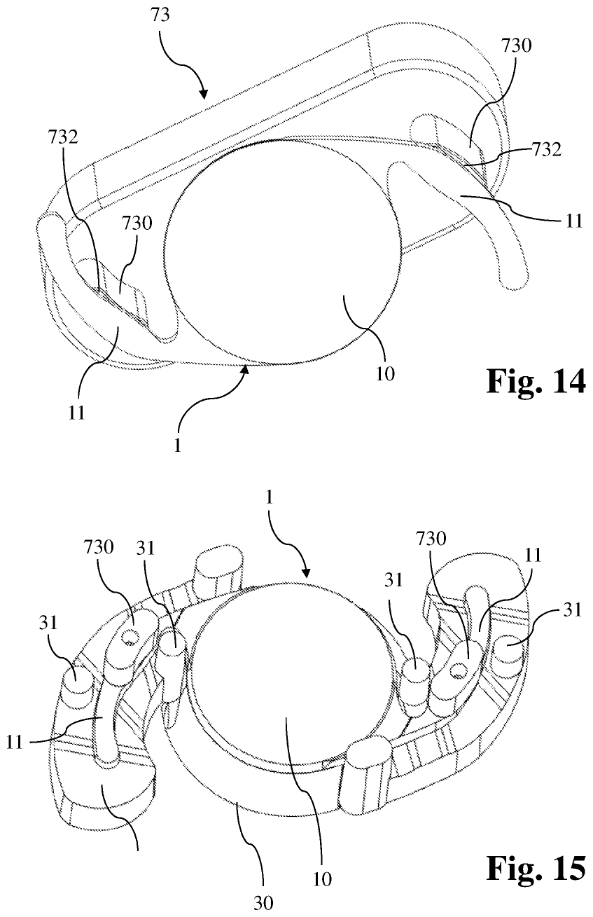

To grip an IOL 1 the haptics 11 of which are regularly arranged on the lens carrier 2 of the first type as is shown on the left-hand side of FIG. 1, the gripper 7 is moved towards the IOL 1 until the lens attachment surfaces 732 of the aspiration ports 730 are positioned adjacent to the haptics 11 of the IOL 1, i.e. a small distance (for example in the range of 0.05 to 0.5 mm, preferably about 0.15 mm) above the haptics 11. Vacuum is then supplied through the pressure supply connectors 72 and through the pressure-tight fluid channels to the aspiration openings 731 of the aspiration ports 730, thus gripping the IOL 1 (picking it up) from the lens carrier 2 by sucking the haptics 11 of the IOL 1 against the lens attachment surfaces 732 of the aspiration ports 730. This state with the haptics 11 of the IOL 1 being attached to the lens attachment surfaces 732 is shown in FIG. 14 (only the gripper end piece 73 of the gripper 7 being shown there). In case an IOL 1 is gripped, this can be detected as the vacuum actually applied increases compared to no IOL 1 being gripped.

Gripper 7 with the IOL 1 attached thereto is then moved to the destination location, for example to a location above the lens carrier 3 of the second type shown in FIG. 1. Both the start location and the destination location have been precisely determined with the aid of the camera 52 (see FIG. 4) so that the apparatus 54 exactly knows the location where the IOL 1 is to be picked up (and in particular also precisely knows the locations of the haptics 11 of the IOL 1) as well as the location where the IOL 1 is to be detached from the gripper 7 (i.e. the location of the lens carrier 3 and the pins 31 and inspection opening 30). This is illustrated in FIG. 15 schematically showing the two aspiration ports 730 of gripper 7 as well as portions of the carrier 3 on which the haptics 11 of the IOL 1 rest as well as the pins 31 for holding the IOL 1 in position. To detach the IOL 1 from the gripper, overpressure is supplied through the pressure supply connectors 72 and through the pressure-tight fluid channels to the aspiration openings 731 of the aspiration ports 730. The alternative supply of either vacuum (during gripping and transportation of the IOL 1) or overpressure (during detachment of the IOL 1) is controlled by the control valves 53 (see FIG. 4).

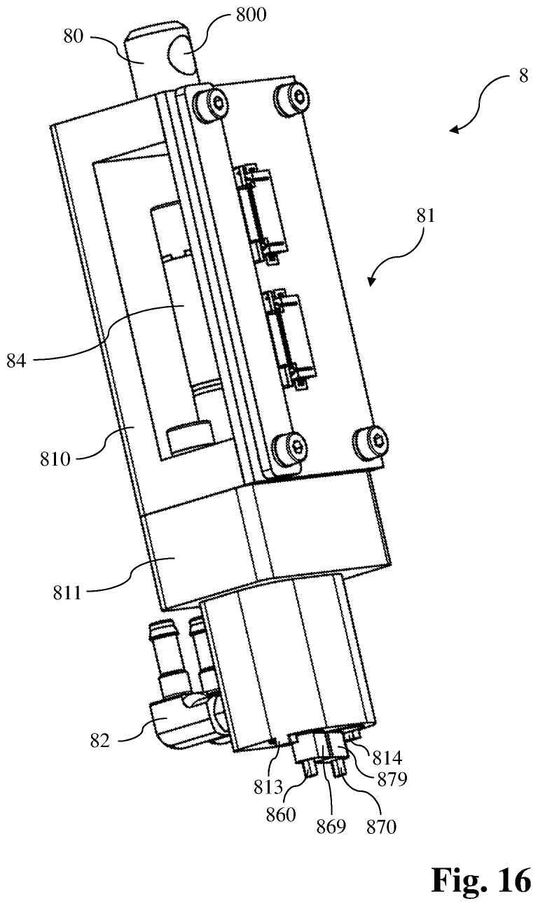

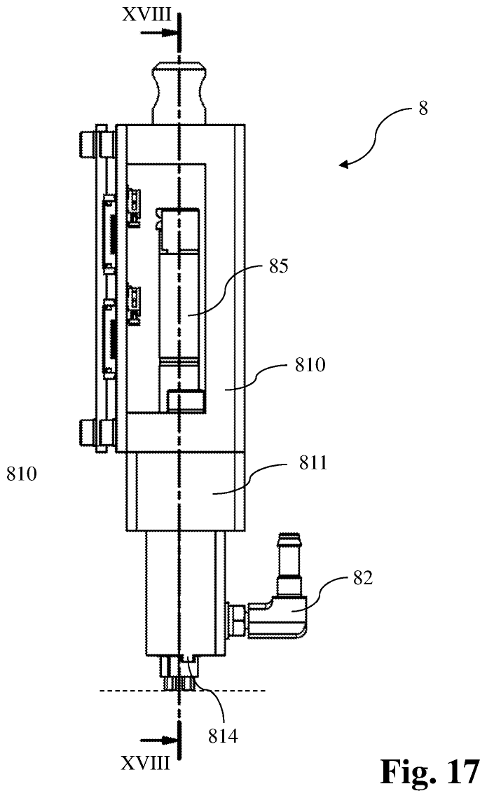

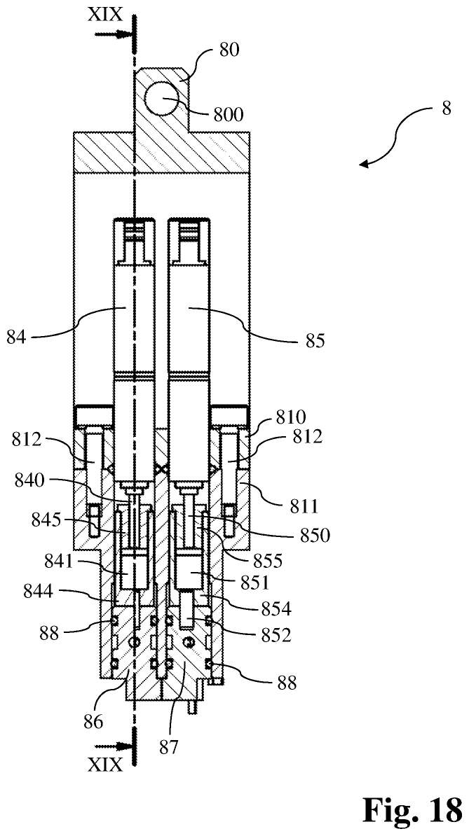

A second embodiment of the gripper 8 of the apparatus 5 according to the invention is described in the following with reference to FIG. 16-FIG. 28. One major difference of this second embodiment of the gripper 8 when compared to the first embodiment of the gripper 7 (see FIG. 5-FIG. 15) is that in this second embodiment of the gripper 8 the (hollow) aspiration ports 860 and 870 are rotatably arranged about a predetermined rotational axis, as will be described in more detail below. As can be seen in the perspective view of the gripper 8 shown in FIG. 16 and in the side view of the gripper 8 shown in FIG. 17, gripper 8 comprises a mounting stud 80 having a through-hole 800 extending transversely through the mounting stud 80, so that the gripper 8 as a whole may be mounted to the mounting shaft 58 of the apparatus 5 with the aid of the fitting screw 581 extending through a through-hole 580 provided in the mounting shaft of the apparatus 5 (see FIG. 6) as well as through the through-hole 800 of the mounting stud 80 of the gripper 8. Gripper 8 further comprises a gripper body 81 having an upper body portion 810 and a lower body portion 811 which are attached to one another with the aid of screws 812 (see FIG. 18). Gripper 8 further comprises two pressure supply connectors 82, each for the alternative supply of vacuum or overpressure to the aspiration ports (similar to the first embodiment of the gripper).

Moreover, gripper 8 comprises two independent rotary motors, a first rotary motor 84 and a second rotary motor 85. As can be seen best in FIG. 18, the first rotary motor 84 comprises a rotary drive shaft 840 and the second rotary motor 85 comprises a rotary drive shaft 850. The rotary drive shaft 840 of the first rotary motor 84 is connected with a first gripper finger 86 through a torque-proof connector, and the rotary drive shaft 850 of the second rotary motor 85 is connected with a second gripper finger 87 through a further torque-proof connector.

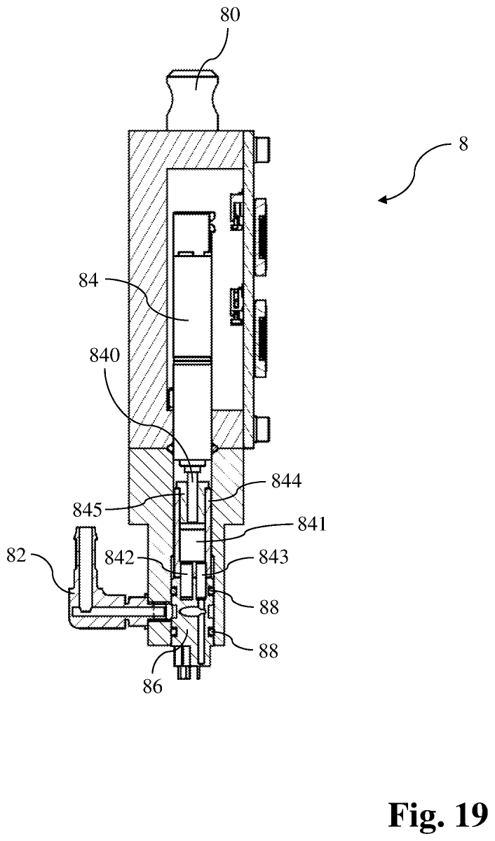

The torque-proof connector connecting the rotary drive shaft 840 of the first rotary motor 84 with the first gripper finger 86 comprises a magnetic clutch. This magnetic clutch comprises a permanent magnet 841 which is mounted to a distal end of the rotary drive shaft 840 in a torque-proof manner (e.g. glued), as well as two pins 842, 843 (see FIG. 19) which are made of a magnetically susceptive material, for example magnetically susceptive stainless steel. The permanent magnet 841 is arranged in a drive bushing 844 which is mounted in a torque-proof manner to a sleeve 845 which in turn is mounted to the drive shaft 840 of the first rotary motor 84 in a torque-proof manner. The two pins 842, 843 extend through respective openings in the distal end face of the drive bushing 844 and are press-fitted into respective blind bores 862, 863 in the proximal end face of first gripper finger 86 (see also FIG. 22). To ensure proper mounting, the two pins 842, 843 may have a different diameter. By way of example, gripper finger 86 may be made from a rigid, durable plastic material. The torque-proof connector thus makes sure that once the first rotary motor 84 drives the rotary drive shaft 840, the first gripper finger 86 is rotated, too.

Similarly, the further torque-proof connector connecting the second rotary drive shaft 850 of the second rotary motor 85 with the second gripper finger 87 comprises a magnetic clutch. This magnetic clutch again comprises a permanent magnet 851 which is mounted to a distal end of the second drive shaft 850 of the second rotary motor 85 in a torque-proof manner (e.g. glued) as well as two pins 852, 853 made of a magnetically susceptive material (only pin 852 visible in FIG. 18), for example magnetically susceptive stainless steel. The permanent magnet 851 is arranged in a drive bushing 854 which is mounted in a torque-proof manner to a sleeve 855 which in turn is mounted to the drive shaft 850 of the second rotary motor 85 in a torque-proof manner. The two pins 852, 853 extend through respective openings in the distal end face of a drive bushing 854 and are press-fitted into respective blind bores in the proximal end face of the second gripper finger 87 (similar to the blind bores 862, 863 of the first gripper finger 86 shown in FIG. 22). Also here, to ensure proper mounting the two pins 852, 853 may have a different diameter. Second gripper finger 87 may also be made from a rigid, durable plastic material. The further torque-proof connector thus makes sure that once the second rotary motor 85 drives the rotary drive shaft 850, the second gripper finger 87 is rotated, too.

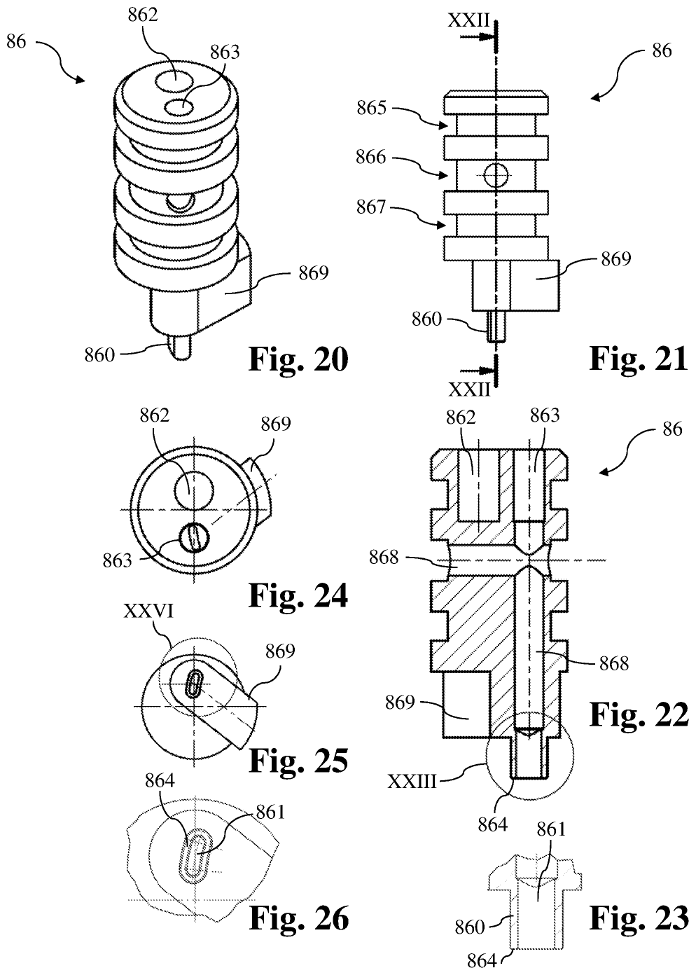

The first gripper finger 86 and the second gripper finger 87 are similarly structured, so that in the following only the first gripper finger 86 will be explained in more detail with the aid of FIG. 20-FIG. 26. First gripper finger 86 comprises the aspiration port 860 arranged at the distal end of first gripper finger 86 (with aspiration port 860 being arranged eccentrically relative to the rotational axis of the gripper finger 86). Aspiration port 860 comprises an aspiration opening 861 which is surrounded by a lens attachment surface 864 (these elements are of a similar structure as those of the first embodiment of the gripper). Gripper finger 86 further comprises blind bores 862 and 863 for the accommodation of the pins 842 and 843 of the magnetic clutch (see FIG. 19), as already explained above. Gripper finger 86 further comprises three grooves, an upper groove 865, a middle groove 866, and a lower groove 867. Upper groove 865 and lower groove 867 are each dedicated to receive an O-ring 88 (see FIG. 18 and FIG. 19) while the middle groove 866 remains empty. Thus, a pressure-tight arrangement of the first gripper finger 86 within the lower body portion 811 of gripper body 81 is achieved while at the same time the first gripper finger 86 is rotatable within the lower body portion 811 of gripper body 81.

Rotation of the first gripper finger 86 may occur around a predetermined first rotational axis (that corresponds to the line XIX-XIX of FIG. 18) which coincides with the central longitudinal axis of the rotary shaft 840 of the first rotary motor 84. This holds for the second gripper finger 87 in a similar manner which is rotatable about the central longitudinal axis of the rotary shaft 850 of the second rotary motor 85. Both rotational axes are normal to a plane defined by the lens attachment surfaces 864 and 874 of the aspiration ports 860 and 861, this plane being indicated in FIG. 17 by the dashed line, the plane extending normal to the plane of the drawing.

From the bottom (radially innermost surface) of the middle groove 866 a fluid channel 868 extends radially into the interior of the gripper finger 86 and down to the aspiration opening 861 of aspiration port 860. Due to the pressure-tight arrangement of the first gripper finger 86 within the lower body portion 811 of gripper body 81, vacuum or overpressure supplied through the pressure supply connector 82 is supplied through a fluid channel that fluidically connects the pressure supply connector 82 and the aspiration opening 861 of the aspiration port 860, this fluid channel being formed through the middle groove 866 and through fluid channel 868 which extends down to the aspiration opening 861 of aspiration port 860.

First gripper finger 86 further comprises an abutment flange 869 arranged immediately proximal to the aspiration port 860. As can be seen best in FIG. 16, the gripper 8 at the distal end thereof (or to be more precise: at the distal end of the lower body portion 811 of gripper body 81) comprises first and second abutment projections 813 and 814 (one for the abutment flange 869 of first gripper finger 86 and the other one for the abutment flange 879 of the second gripper finger 87). The first and second abutment projections 813 and 814 project distally from the distal end of the lower body portion 810, and thus from the distal end of the gripper body 81. These first abutment projection 813 forms a stop for the abutment flange 869 of the first gripper finger 86, and the second abutment projection 814 forms a stop for the abutment flange 879 of the second gripper finger 87. Thus, the first abutment projection 813 defines a predetermined rotational orientation of the aspiration port 860 of the first gripper finger 86 when the abutment flange 869 abuts against the first abutment projection 813, and the second abutment projection 814 defines a predetermined rotational orientation of the aspiration port 870 of the second gripper finger 87 when the abutment flange 879 abuts against the second abutment projection 814. Therefore, the first and second abutment projections 813 and 814 may serve as a reference rotational position for the first and second gripper fingers 86 and 87 to which these can be rotated during a reference run. From this reference rotational position, the first and second gripper fingers 86 and 87 can be rotated to the desired rotational position.

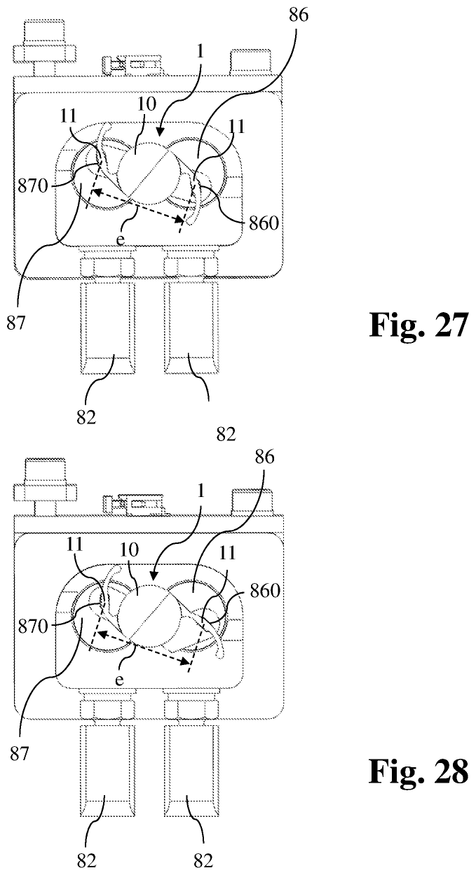

The gripper 8 is of course suitable to reliably pick up an IOL 1 the haptics 11 of which are regularly oriented (i.e. the haptics 11 are arranged as this is shown for the IOL 1 arranged on lens carrier 2 on the left-hand side in FIG. 1). However, the gripper 8 is also particularly suitable to reliably pick up an IOL 1 the haptics 11 of which are irregularly oriented (i.e. the haptics 11 are bent or flexed, as this is shown for the IOL 1 arranged on the lens carrier 2 on the right-hand side in FIG. 1). This is described in the following with the aid of FIG. 27 and FIG. 28.

FIG. 27 shows the IOL 1 with the haptics 11 thereof attached to the lens attachment surfaces of the aspiration port 860 of first gripper finger 86 and of the aspiration port 870 of the second gripper finger 87. In this case, the haptics 11 of the IOL 1 are regularly oriented as this is shown for the IOL 1 on lens carrier 2 on the left-hand side in FIG. 1. When the camera 52 has captured the image of the IOL 1 an image analysis is performed, and from this image analysis the rotational orientation of the haptics 11 of the IOL 1 is determined. If the haptics 11 are regularly oriented, a control unit (not shown) coupled to the camera 52 and to the gripper 8 moves gripper 8 to the location (in the x-y plane) where the IOL 1 is located. The first gripper finger 86 is rotated to the rotational orientation shown in FIG. 27 in which the aspiration port 860 of the first gripper finger 86 has the required orientation that corresponds to the orientation of the associated haptic 11 of IOL 1. To achieve this, the control unit drives the first rotary motor 84 whereby the first gripper finger 86 is rotated (via the magnetic clutch as described in detail above) until the aspiration port 860 has the required orientation that corresponds to the orientation of the associated haptic 11. Similarly, the control unit drives the second rotary motor 85 whereby the second gripper finger 87 is rotated (again via the magnetic clutch as described in detail above) until the aspiration port 870 has the required orientation that corresponds to the orientation of the associated haptic 11. In this orientation, the distance e between the two aspiration ports 860 and 870 may be about 9.8 mm; this distance is in any event larger than the diameter of the optical body 10 of IOL 1 to be gripped, so that the IOL 1 may only be gripped at the haptics 11). Generally, this distance e ranges from 5 mm to 17 mm, and more preferably distance e ranges from 8 mm to 12 mm. So once the camera 52 (see FIG. 4) has taken an image of the IOL 1 arranged on the carrier, the center of the optical body 10 of the IOL 1 may be determined with the aid of image processing software and the rotational orientation of the haptics 11 is then also determined. The first and second gripper fingers 86 and 87 are then rotated to the rotational orientation of the haptics 11.

As can be seen, in this embodiment the distance e is not fixed and may vary depending on the rotational orientation of aspiration port 860 and aspiration port 870. The gripper 8 is then lowered until the aspiration port 860 and the aspiration port 870 are each arranged a short distance (e.g. 0.05 mm to 0.5 mm, preferably 0.15 mm) above the associated haptic 11.

Vacuum is then supplied to the pressure supply connectors 82 with the aid of the control valves 53, so that the haptics 11 of the IOL 1 are sucked against the lens attachment surface of the aspiration port 860 and to the lens attachment surface of the aspiration port 870, respectively, thus picking the IOL 1 up. In case an IOL 1 is gripped, this can be detected as the vacuum actually applied increases compared to no IOL 1 being gripped. The gripper 8 with the IOL 1 attached thereto (the IOL 1 being gripped only at the haptics 11) is then raised again, and is subsequently moved to the location (in the x-y plane) of the lens carrier 3 (FIG. 1). The gripper 8 is then lowered again until the aspiration port 860 and the aspiration port 870 are arranged a short distance above the carrier 3. The IOL 1 is then detached from the gripper 8 through the supply of overpressure to the pressure supply connectors 82, this supply again being controlled by the control valves 53.

This can be performed in the same manner for an IOL 1 the haptics 11 of which are arranged at an irregular orientation as shown for the IOL 1 arranged on the lens carrier 2 on the right-hand side in FIG. 1. This is evident from FIG. 28. Due to the orientation of the haptics 11 of the IOL 1 arranged on the lens carrier 2 on the right-hand side in FIG. 1 being different from the regular orientation of the haptics 11 of the IOL 1 arranged on the lens carrier 2 on the left-hand side in FIG. 1, the first gripper finger 86 and the second gripper finger 87 of gripper 8 must be rotated to a different rotational orientation (i.e. to the irregular rotational orientation) such that the aspiration port 860 of the first gripper finger 86 and the aspiration port 870 of the second gripper finger 87 have the required orientation that corresponds to the orientation of the associated haptic 11 of the IOL 1 arranged on the lens carrier 2 shown on the right-hand side in FIG. 1. This is again performed with the aid of the camera 52 capturing the image of the IOL 1 with the haptics 11 arranged at the irregular orientation, and through an image analysis performed for determining the irregular rotational orientation of the haptics 11 of IOL 1. As can be seen, in this orientation the distance e between the two aspiration ports 860 and 870 may be about 9.8 mm. Once the aspiration port 860 of the first gripper finger 86 and the aspiration port 870 of the second gripper finger 87 have been rotated to the required orientation, the gripper 8 is lowered and vacuum is supplied to the pressure supply connectors 82, so that the haptics 11 of the IOL are sucked against the lens attachment surface of the respective aspiration port, thus picking the IOL 1 up. The gripper 8 with the IOL 1 attached thereto (the IOL 1 again being gripped only at the haptics 11 still having the irregular orientation) is then moved to the lens carrier 3 (FIG. 1). Either after the gripper 8 has been raised, during the movement of the IOL 1 to the lens carrier 3, or at the time the gripper 8 has arrived at the location above the lens carrier 3, but in any event prior to releasing the IOL 1 from the gripper 8, the first gripper finger 86 and the second gripper finger 87 are rotated again such that the haptics 11 of the IOL 1 have the regular orientation. The gripper 8 is then lowered and the IOL 1 detached from the gripper 8 and placed on lens carrier 3 through the supply of overpressure to the supply connectors 82, this supply again being controlled by the control valves 53.

Embodiments of the invention have been described with the aid of the drawings. However, the invention is not limited to these embodiments, but rather many changes and modifications are possible without departing from the teaching underlying the invention. Therefore, the scope of protection is not limited to the embodiments described but rather is defined by the appended claims.

1. Method for the automated transfer of an intraocular lens (1) comprising an optical lens body (10) and two haptics (11) attached to a peripheral edge of the optical lens body (10) and extending outwardly from the peripheral edge of the optical lens body (10), the method comprising the steps of:
picking the intraocular lens (1) up at a start location;
moving the intraocular lens (1) to a destination location;
releasing the intraocular lens (1) at the destination location,
wherein picking the intraocular lens (1) up at the start location comprises
gripping the intraocular lens (1) only at the haptics (11) of the intraocular lens (1).

2. Method according to claim 1, wherein gripping the intraocular lens (1) only at the haptics (11) is performed using a gripper (7; 8) comprising aspiration ports (730; 860, 870) to attach the intraocular lens (1) to the gripper only at the haptics (11) of the intraocular lens (1) by positioning the aspiration ports (730; 860, 870) of the gripper adjacent to the haptics (11) of the intraocular lens (1) and applying vacuum to the aspiration ports (730; 860, 870), and wherein releasing the intraocular (1) lens from the gripper at the destination location is performed by applying overpressure to the aspiration ports (730; 860, 870) to detach the haptics (11) of the intraocular lens (1) from the aspiration ports (730; 860, 870).

3. Method according to claim 2, wherein the aspirations ports (730; 860, 870) of the gripper (7; 8) comprise two aspiration ports (730; 860, 870) arranged at a distal end of the gripper and projecting distally away from the gripper, each of the two aspiration ports (730; 860, 870) comprising at its distal end an aspiration opening (731; 861, 871) surrounded by a lens attachment surface (732; 864), wherein the method further comprises
positioning the distal end of one aspiration port (730; 860) of the two aspiration ports adjacent to one of the two haptics (11) of the intraocular lens (1) such that the aspiration opening (731; 861) of that one aspiration port (730; 860) is covered by a portion of the one of the two haptics (11) arranged adjacent thereto,
positioning the other aspiration port (730; 870) of the two aspiration ports adjacent to the other of the two haptics (11) of the intraocular lens (1) such that the aspiration opening (730; 871) of that other aspiration port (730; 870) is covered by a portion of the other one of the two haptics (11), and
applying the vacuum to the aspiration openings (731; 861, 871) of the two aspiration ports to attach the one of the two haptics (1) to the lens attachment surface (732, 864) of the one aspiration port (730; 860) and the other of the two haptics (11) to the lens attachment surface (732; 874) of the other aspiration port (730; 870).

4. Method according to claim 3, wherein the gripper (7) is a gripper having the two aspiration ports (730) fixedly arranged at the distal end of the gripper (7).

5. Method according to claim 3, wherein the gripper (8) is a gripper having the two aspiration ports (860, 870) rotatably arranged at the distal end of the gripper, each of the two aspiration ports (860) being rotatably arranged about a respective predetermined rotational axis which is normal to a plane defined by the lens attachment surfaces (864, 874) of the two aspiration ports (860, 870).

6. Method according to claim 5, wherein the method further comprises
prior to applying the vacuum to the aspiration openings (861, 871) of the two aspiration ports (860, 870), determining an actual rotational orientation of each of the two haptics (11) of the intraocular lens (1),
rotating each of the two aspiration ports (860, 870) about the respective predetermined rotational axis until the rotational orientation of the one aspiration port (860) corresponds to the determined actual rotational orientation of the one of the two haptics (11) and the rotational orientation of the other aspiration port (870) corresponds to the determined actual rotational orientation of the other of the two haptics (11), and thereafter
applying the vacuum to the aspiration openings (861, 871) of the two aspiration ports to attach the one of the two haptics (11) to the lens attachment surface (864) of the one aspiration port (860) and the other of the two haptics (11) to the lens attachment surface (874) of the other aspiration port (870).

7. Method according to claim 6, wherein the method further comprises
in case the actual rotational orientation of the one of the two haptics (11) or of the other of the two haptics (11) of the intraocular lens (1) deviates from a respective predetermined rotational orientation:
rotating the one aspiration port (860) with the one of the two haptics (11) attached to the lens attachment surface (864) thereof and/or the other aspiration port (870) with the other of the two haptics (11) attached to the lens attachment surface (874) thereof about the respective predetermined rotational axis until each of the two haptics (11) has the predetermined rotational orientation, and
at the destination location, applying the overpressure to the aspiration openings (861, 871) of the two aspiration ports (860, 870) with each of the two haptics (11) having the predetermined rotational orientation.

8. Apparatus (5) for the automated transfer of an intraocular lens (1) comprising an optical lens body (10) having a peripheral edge as well as two haptics (11) attached to the peripheral edge of the optical lens body (10) and extending outwardly from the peripheral edge of the optical lens body (10), the apparatus comprising a gripper (7; 8) including:
pressure supply connectors (72; 82) for the supply of vacuum or overpressure;
two aspiration ports (730; 860, 870) arranged at a distal end of the gripper and projecting distally away from the gripper, each of the two aspiration ports (730; 860, 870) at a distal end thereof comprising an aspiration opening (731; 861, 871) that is surrounded by a lens attachment surface (732; 864, 874),
two separate fluid channels (712, 734, 735; 866, 868), each of the two separate fluid channels fluidically connecting a respective one of the two aspiration ports with the pressure supply connectors (72; 82) for the supply of vacuum or overpressure so as to form two separate fluidic connections between the pressure supply connectors (72; 82) and the aspiration openings (731; 861, 871) of the aspiration ports (730; 860, 870),
wherein the two aspiration ports are spaced from one another by a distance (d; e) ranging from 5 mm to 17 mm measured in a plane defined by the lens attachment surfaces (732; 864, 874), for gripping the intraocular lens (1) only at the haptics (11) by attaching the haptics (11) of the intraocular lens to the lens attachment surfaces (732; 864, 874) of the aspiration ports (730; 860, 870), and for releasing the intraocular lens (1) by detaching the haptics (11) of the intraocular lens (1) from the lens attachment surfaces (732; 864, 874).

9. Apparatus according to claim 8, wherein the two aspiration ports (730) are fixedly arranged at the distal end of the gripper (7).

10. Apparatus according to claim 8, wherein the two aspiration ports (860, 870) are rotatably arranged at the distal end of the gripper (8), each of the two aspiration ports (860, 870) being rotatably arranged about a respective predetermined rotational axis which is normal to the plane defined by the lens attachment surfaces (864, 874).

11. Apparatus according to claim 10, wherein the gripper further comprises two independent rotary motors, a first rotary motor (84) and a second rotary motor (5) each having a rotary drive shaft (840, 850), and wherein the rotary drive shaft (840) of the first rotary motor (84) is connected by a torque-proof connector with a first gripper finger (86) having one of the two aspiration ports (860) arranged at a distal end thereof, and wherein the rotary drive shaft (850) of the second rotary motor (5) is connected by a further torque-proof connector with a second gripper finger (87) having the other one of the two aspiration ports (870) arranged at a distal end thereof.

12. Apparatus according to claim 11, wherein each of the torque-proof connector and the further torque-proof connector comprises a magnetic clutch including a permanent magnet (841, 851) and two pins (842, 843, 852, 853) made of a magnetically susceptive material, the permanent magnet (841, 851) being mounted in a torque-proof manner to a distal end of the rotary drive shaft (840, 850) and facing towards a proximal end of the respective one of the first or second gripper fingers (86, 87), and the two pins (842, 843, 852, 853) being arranged at the proximal end of the respective one of the first or second gripper fingers (86, 87) and facing towards the permanent magnet (841, 851), with the distal ends of the two pins (842, 843, 852, 853) being fixedly connected with the respective one of the first or second gripper fingers (86, 87), and with the proximal ends of the two pins (842, 843, 852, 853) being magnetically coupled to the permanent magnet (841, 851).

13. Apparatus according to claim 11 or claim 12, wherein the first gripper finger (86) comprises an abutment flange (869) arranged immediately proximal to the one aspiration port (860) and the second gripper finger (87) comprises a further abutment flange (879) arranged immediately proximal to the other aspiration port (870), and wherein the gripper (8) at its distal end comprises first and second abutment projections (813, 814) projecting distally from the distal end of the gripper at opposite sides of the gripper, the first abutment projection (813) forming a stop for the abutment flange (869) of the first gripper finger (86) to define a predetermined rotational orientation of the one aspiration port (860) when the abutment flange abuts against the first abutment projection (813), and the second abutment projection (814) forming a stop for the further abutment flange (879) of the second gripper finger (87) to define a predetermined rotational orientation of the other aspiration port (870) when the further abutment flange (879) abuts against the second abutment projection (814).

14. Apparatus according to any one of claims 8 to 13, further comprising an illumination source (51) for illuminating an intraocular lens (1) carried by a lens carrier (2) as well as a camera (52) for capturing an image of the illuminated intraocular lens (1) carried by the lens carrier (2) to determine the position of the intraocular lens and the rotational orientation of the haptics (11) of the intraocular lens (1) carried by the lens carrier (2), and further comprising a control unit coupled to the camera (52) and to the gripper (7, 8), for moving the gripper (7, 8) with the two aspiration ports (730; 860, 870) to a position in which the two aspiration ports (730; 860, 870) are arranged adjacent to the haptics (11) of the intraocular lens (1) such that the rotational orientation of the two aspiration ports (730; 860, 870) corresponds to the determined actual rotational orientation of the two haptics (11) of the intraocular lens (1).

15. Apparatus according to claim 14, further comprising a support plate (50) comprising a plurality of mounting locations (500) for mounting different types of lens carriers (2, 3) thereto, and further comprising at least two different lens carriers (2, 3) of different types mounted to the mounting locations (500), wherein the support plate (50), the mounting locations (500) and the at least two lens carriers (2, 3) of the different types are configured such that an intraocular lens arranged on a said lens carrier (2, 3), regardless of the type of lens carrier, is arranged in the same plane parallel to the plane defined by the lens attachment surfaces (732; 864, 874) of the aspiration ports (730; 860, 870).
